(19) United States
(12) Patent Application Publication
(10) Pub. No.: US 2020/0337987 A1
(43) Pub. Date: Oct. 29, 2020

(54) Packaged Probiotic Composition and Methods

(71) Applicant: Tempo Probiotics LLC, Plymouth, MI (US)

(72) Inventors: David James O'Hagan, Whitmore Lake, MI (US); Savita Sunil Nikam, Canton, MI (US)

(73) Assignee: Tempo Probiotics LLC, Plymouth, MI (US)

(21) Appl. No.: 16/395,882

(22) Filed: Apr. 26, 2019

(51) Int. Cl. A61K 8/99; B65D 83/68; A61Q 19/00

(52) U.S. Cl. CPC A61K 8/99; B65D 83/682; A61K 2800/31; A61K 2800/70; A61K 2800/87; A61Q 19/00

(57) ABSTRACT

Various packaged probiotic compositions, methods of producing a packaged probiotic composition, and cosmetic methods are described. A packaged probiotic composition includes a storage vessel that defines a first chamber, a second chamber, and a wall that separates the first and second chambers. The first chamber includes a lyophilized probiotic bacteria matrix and the second chamber includes a moisture-providing composition. A dispensing mechanism is individually coupled to the first and second chambers and, when activated, draws pre-measured portions of the lyophilized probiotic bacteria matrix and the moisture-providing composition from the respective first and second chambers, mixes the pre-measured portions, and dispenses the resulting mixed composition.

### FIELD

The disclosure relates to the field of probiotic compositions. More particularly, the disclosure relates to the field of probiotic skin care compositions. Specific examples relate to packaged probiotic compositions. The disclosure also relates to methods of producing a packaged probiotic composition and cosmetic methods.

### BACKGROUND

Various probiotic compositions are known in the art. For example, some conventional probiotic compositions are used for treating skin. These probiotic compositions have several drawbacks, however. For example, these probiotic compositions are typically administered orally or include water in the composition, which may affect the probiotic composition during use. Furthermore, various probiotic composition dispensers are known in the art. For example, some conventional probiotic composition dispensers contain either one or various forms of compositions, including a probiotic composition. These probiotic composition dispensers have several drawbacks, however. For example, the conventional probiotic composition dispensers are packaged in a way that the probiotic composition dispenser does not mix the compositions if the dispenser contains two compositions, which often necessitates additional mixing by the user after dispensing.

A need exists, therefore, for improved packaged probiotic compositions, methods of producing a packaged probiotic composition, and cosmetic methods.

### BRIEF SUMMARY OF SELECTED EXAMPLES

Various example packaged probiotic compositions are described herein.

An example packaged probiotic composition includes a storage vessel and a dispensing mechanism. The storage vessel defines a first chamber, a second chamber, and a wall that separates the first and second chambers. The dispensing mechanism is individually coupled to the first and second chambers. The first chamber includes a lyophilized probiotic bacteria matrix, and the second chamber includes a moisture-providing composition. During activation, the dispensing mechanism draws pre-measured portions of the lyophilized probiotic bacteria matrix and the moisture-providing composition from the respective first and second chambers, mixes the pre-measured portions, and dispenses the resulting mixed composition.

Another example packaged probiotic composition includes a storage vessel and a dispensing mechanism. The storage vessel defines a first chamber, a second chamber, and a wall that separates the first and second chambers. Additionally, each of the first and second chambers are substantially free of air. The dispensing mechanism is individually coupled to the first and second chambers. The first chamber includes a lyophilized probiotic bacteria matrix, and the lyophilized probiotic bacteria matrix includes the bacteria Lactobacillus rhamnosus. The second chamber includes a moisture-providing composition. During activation, the dispensing mechanism draws pre-measured portions of the lyophilized probiotic bacteria matrix and the moisture-providing composition from the respective first and second chambers, mixes the pre-measured portions, and dispenses the resulting mixed composition.

Another example packaged probiotic composition includes a storage vessel and a dispensing mechanism. The storage vessel defines a first chamber, a second chamber, and a wall that separates the first and second chambers. Additionally, each of the first and second chambers are substantially free of air. The dispensing mechanism is individually coupled to the first and second chambers. The first chamber includes a lyophilized probiotic bacteria matrix, and the lyophilized probiotic bacteria matrix includes the bacteria Lactobacillus rhamnosus with the compounds of dimethicone, polysilicone-11, and butyrospermum parkii butter. The second chamber includes a moisture-providing composition, and the moisture-providing composition includes at least Vitamin A, E, C and K, Hyaluronic acid, Biotin, Niacinamide, Resveratrol, dimethylaminoethanol, PCA, Palmitoyl Tripeptide-1, Palmitoyl Tetrapeptide-7, Ceramides, Alpha-Lipoic Acid, Malic Acid, and Coenzyme Q10. During activation, the dispensing mechanism draws pre-measured portions of the lyophilized probiotic bacteria matrix and the moisture-providing composition from the respective first and second chambers, mixes the pre-measured portions, and dispenses the resulting mixed composition.

Various methods of producing a packaged probiotic composition are also described herein.

Various cosmetic methods are also described herein.

### DESCRIPTION OF FIGURES

FIG. 1 is a front view of an example packaged probiotic composition.

FIG. 2 is an exploded view of the packaged probiotic composition illustrated in FIG. 1.

FIG. 3 is a top view of the packaged probiotic composition illustrated in FIG. 1.

FIG. 4 is a side view of the packaged probiotic composition illustrated in FIG. 1.

FIG. 5 is a bottom view of the dispenser head of the packaged probiotic composition illustrated in FIG. 1.

FIG. 6 is a front view of the dispenser head of the packaged probiotic composition illustrated in FIG. 1.

FIG. 7 is an exploded view of another example packaged probiotic composition.

FIG. 8 is an exploded view of another example packaged probiotic composition.

FIG. 9 is a flowchart representation a method of producing a packaged probiotic composition.

FIG. 10 is a flowchart representation a cosmetic method.

### DETAILED DESCRIPTION OF SELECTED EXAMPLES

The following detailed description and the appended drawings describe and illustrate various example packaged probiotic compositions, methods of producing a packaged probiotic composition, and cosmetic methods. The description and drawings are provided to enable one skilled in the art to make and use one or more packaged probiotic compositions and to perform the example methods. They are not intended to limit the scope of the claims in any manner.

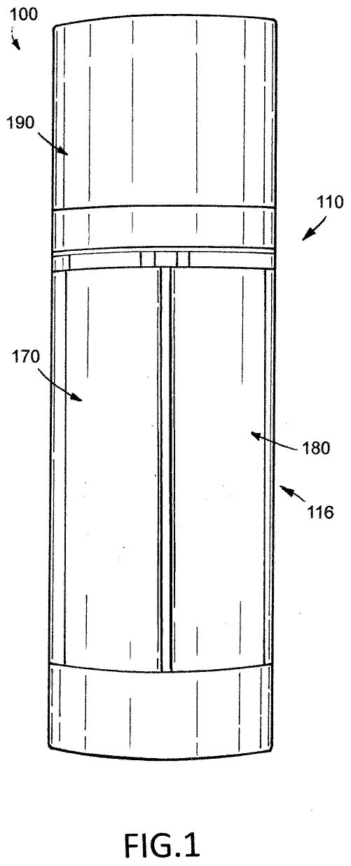

FIGS. 1 through 6 illustrate an example packaged probiotic composition 100. The packaged probiotic composition 100 has a storage vessel 110, a cap 190, and a dispensing mechanism 200.

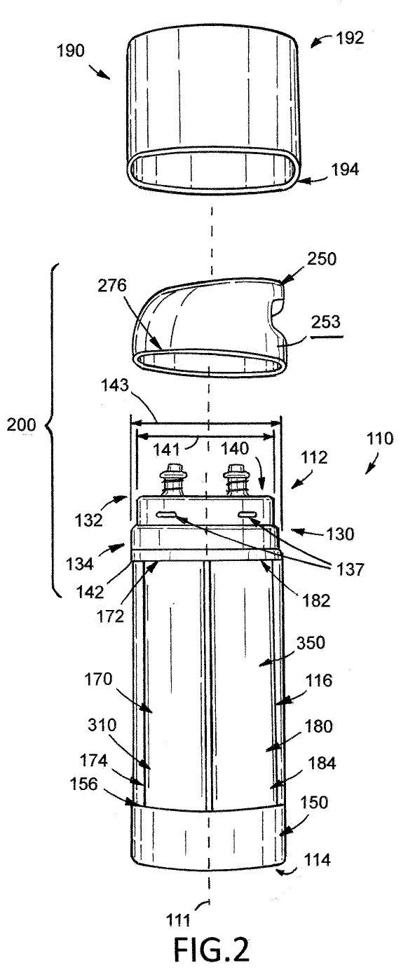

The storage vessel 110 has a storage vessel first end 112, a storage vessel second end 114, a lengthwise axis 111 that extends between the storage vessel first and second ends 112, 114, a main body 116, a first stopper 130, a second stopper 150, a first chamber 170, and a second chamber 180. As best illustrated in FIG. 2, the first stopper 130 is located toward the storage vessel first end 112, the main body 116 is located between the storage vessel first and second ends 112, 114, and the second stopper 150 is located toward the storage vessel second end 114. The first and second chambers 170, 180 are located between the storage vessel first and second ends 112, 114, which are attached and secured to each of the first and second stoppers 130, 150.

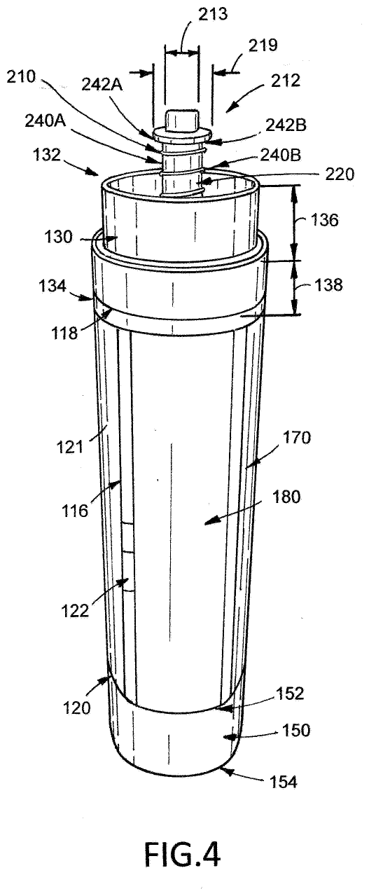

As illustrated in FIG. 4, the main body 116 has a main body first end 118, a main body second end 120, a main body outer surface 121, and a main body inner surface (not illustrated), and a main body chamber 122. As best illustrated in FIG. 4, the main body first end 118 is sized and configured to interface and attach the first stopper second end 134, which is described in detail below. Additionally, the main body second end 120 is sized and configured to interface and attach the second stopper first end 152, which is described in detail below. Furthermore, the main body chamber 122 is sized and configured to dispose each of the first and second chambers 170, 180 between the main body first and second ends 118, 120.

As illustrated in FIGS. 2, 3, and 4, the first stopper 130 has a first stopper first end 132 and a first stopper second end 134. In the illustrated embodiment, the first stopper 130 has a first stopper first portion 136 and a first stopper second portion 138. The first stopper first portion 136 extends from the first stopper first end 132 to the medial portion of the first stopper 130. The first stopper first portion 136 defines a first stopper first opening 140 that extends from the first stopper first end 132 toward the first stopper second end 134 which terminates at the medial portion of the first stopper 130. In addition, the first stopper first opening 140 defines a first opening diameter 141. Similarly, the first stop second portion 138 extends from the first stopper second end 134 to the medial portion of the first stopper 130. The first stopper second portion 138 defines a first stopper second opening 142 that extends from the first stopper second end 134 toward the first stopper first end 132 which terminates at the medial portion of the first stopper 130. In addition, the first stopper second opening 142 defines a second opening diameter 143. In the illustrated embodiment, the first stopper first and second openings 140, 142 lie on the same plane, but directly oppose each other. Furthermore, the first opening diameter 141 is greater than the second opening diameter 143. The first stopper first and second portions 136, 138 are considered advantageous at least because the first stopper first portion 136 is sized and configured to interface and receive the dispensing head 250 to operate the dispensing mechanism 200, which is described in detail below, and the first stopper second portion 138 is sized and configured to interface and receive the main body first end 118.

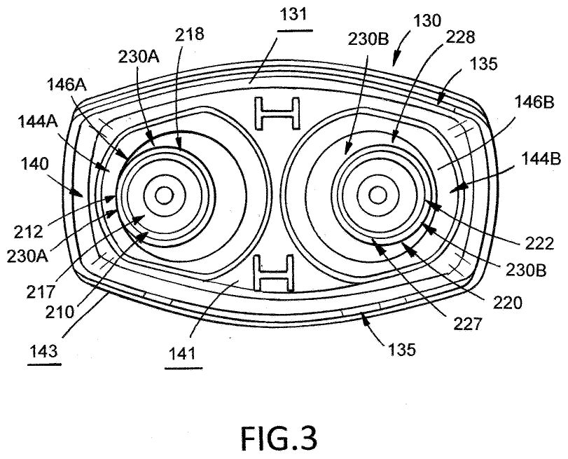

As illustrated in FIG. 3, the first stopper 130 also defines first and second cavities 144A, 144B that define first and second openings 146A, 146B. Each of the first and second cavities 144A, 144B extends from the medial portion of the first stopper 130 into the first stopper first portion 136 toward the first stopper first end 132. Each of the first and second cavities 144A, 144B of the first stopper 130 are considered advantageous at least because each of the first and second cavities 144A, 144B adequately holds each of the first and second chambers 170, 180 inside of the storage vessel 110. Additionally, each of the first and second cavities 144A, 144B defines the first and second openings 146A, 146B. The first and second openings 146A, 146B are sized and configured to interface and receive first and second tubes 210, 220 of the dispensing mechanism 200. Each of the first and second openings 146A, 146B also extends away from the each of the first and second cavities 144A, 144B at a length (not illustrated) in which each of the first and second openings 146A, 146B defines a threading (not illustrated) that is complementary to each of the first and second collars 230A, 230B of the dispensing mechanism. Each of the first and second openings 146A, 146B of the first and second cavities 144A, 144B is considered advantageous at least because the each of the first and second openings 146A, 146B allows the dispensing mechanism 200 to function properly by securing the first and second tubes 210, 220 to the first stopper 130 in which the dispensing mechanism 200 can properly dispense both first and second substances 310, 350 from the first and second chamber 170, 180

As illustrated in FIGS. 2, 3 and 4, the first stopper 130 also defines a first stopper first inner surface 131, a first stopper second inner surface (not illustrated), and a first stopper outer surface 133. In this embodiment, the first inner stopper first surface 131 is defined by the first stopper first portion 136, and the first stopper second inner surface is defined by the first stopper second portion 138. The first stopper first inner surface 131 interfaces with the dispensing head outer surface 253 such that the first stopper first inner surface 131 receives and secures the dispensing head 250 to the first stopper 130. The attachment between the first stopper first inner surface 131 and the dispensing head 250 is accomplished due to the first stopper first inner surface 131 and the dispensing head 250 having complementary attachment members. Here, the first stopper first inner surface 131 defines a first set of attachment members 135 that are complementary to a second set of attachment members 276 defined by the dispensing head outer surface 253. The first and second set of attachment members 135, 276 of the first stopper first inner surface 131 and the dispensing head 250 are considered advantageous at least because when a user engages the dispensing head 250 during use, the dispensing head 250 is able to move from a first position to a second position without having the dispensing head 250 becoming disengaged from the dispensing mechanism 200. Additionally, the first stopper second inner surface of the first stopper 130 interfaces and engages the main body 116 to attach the first stopper 130 to the main body 116.

Furthermore, the first stopper outer surface 133 is defined by both the first stopper first and second portions 136, 138. As illustrated in FIG. 2, the first stopper outer surface 133 interfaces with the cap 190 such that once the cap 190 is placed onto the storage vessel 110, the cap 190 is attached and secured to the first stopper outer surface 133. The attachment between the first stopper outer surface 133 and the cap 190 is accomplished due to the first stopper outer surface 133 and the cap 190 having complementary attachment members. Here, the first stopper outer surface 133 defines a third set of attachment members 137 that is complementary to a fourth set of attachment members 194 defined by cap 190. The third and fourth attachment members 137, 194 of the first stopper outer surface 133 and the cap 190 are considered advantageous at least because the third and fourth attachment members 137, 194 prevent a user from engaging the dispensing head 250 when not in use due to the cap 190 remaining attached and secured to first stopper 130.

FIGS. 2 and 4 illustrate the second stopper 150, which defines a second stopper first end 152, a second stopper second end 154, a second stopper main opening 156, third and fourth cavities (not illustrated) and third and fourth openings (not illustrated). In the illustrated embodiment, the second stopper main opening 156 extends from the second stopper first end 152 to the second stopper second end 154. The second main opening 156 is sized and configured to interface and receive the main body second end 120 such that the second stopper 150 attaches to the main body 116. Additionally, the second stopper main opening 156 is sized and configured to dispose the third and fourth cavities. Each of the third and fourth cavities is disposed on the second stopper second end 154 and extends away from the second stopper second end 154 toward the second stopper first end 152. Additionally, each of the third and fourth cavities defines each of the third and fourth openings. Each of the third and fourth openings and each of the third and fourth cavities are sized and configured to receive and house the first and second chambers 170, 180.

FIGS. 1, 2, and 4 illustrate the first and second chambers 170, 180. The first chamber 170 has a first chamber first end 172, a first chamber second end 174, and a first chamber length that is measured from the first chamber first end 172 to the first chamber second end 174. The first chamber 170 is disposed parallel to the main body 116 relative to the lengthwise axis 111 of the storage vessel 110. The first chamber 170 contains a first substance 310 therein, such as anhydrous lyophilized probiotic bacteria matrix, which is described in more detail below. Similarly, the second chamber 180 has a second chamber first end 182, a second chamber second end 184, and a second chamber length that is measured from the second chamber first end 182 to the second chamber second end 184. The second chamber 180 is also disposed parallel to the main body 116 relative to the lengthwise axis 111 of the storage vessel 110. The second chamber 180 also contains a second substance 350 therein, such as moisture-providing composition, which is also described in more detail below. Furthermore, each of the first and second chambers 170, 180 of the storage vessel 110 is sufficiently free of air once the first and second substances 310, 350 are disposed into each of the first and second chambers 170, 180.

The separation of the first and second substances 310, 350 in each of the first and second chambers 170, 180 is considered advantageous at least because the first substance 310, such as anhydrous lyophilized probiotic bacteria matrix, must remain separate from the second substance 350, such as moisture-providing composition, in order to prevent and/or mitigate the first substance from absorbing water in any form. Additionally, each of the first and second chambers 170, 180 being sufficiently free of air is also considered advantageous at least because preventing a first and/or second substance 310, 350 to react with the air, such as the anhydrous lyophilized probiotic bacteria matrix, may provide adequate results in which a probiotic compound may promote healing, increase in collagen, and/or reduction in inflammation. Additionally, a substance that may be adversely affected by air, such as a substance that may deteriorate or become less effective from the presence of air over time (e.g., an anhydrous substance), may be contained within the airless first or second chambers 170, 180 to avoid such adverse effects. Skilled artisans, however, will appreciate that the present disclosure is not so limited, as the present disclosure contemplates embodiments having one or more non-airless chambers as well. In such embodiments, those substances that may not be adversely affected by air may then be contained within non-airless chambers.

While the packaged probiotic composition 100 includes first and second chambers 170, 180 that are housed and secured inside the storage vessel 110, the packaged probiotic composition 100 may accept removable first and second chambers 470, 480. Each of the removable first and second chambers 470, 480 are sized and configured to be used in the packaged probiotic composition 100.

As illustrated in FIGS. 2, 3, and 4, the dispensing mechanism 200 includes a first tube 210, a second tube 220, first and second collars 230A, 230B, first and second spring mechanisms 240A, 240B, and a dispensing head 250.

In the illustrated embodiment, the first tube 210 has a first tube first end 212, a first tube second end (not illustrated), a length that is measured from the first tube first end 212 to the first tube second end, a first tube first diameter 213, and a circumferential wall 216 that extends between the first tube first and second ends 212, 214. The first tube 210 also defines a first inlet opening 215 and a first outlet opening 217 in which the first inlet opening (not illustrated) is disposed at the first tube second end, and the first outlet opening 217 is disposed at the first tube first end 212. As described in more detail below, the first inlet opening provides suction such that once the dispensing head 250 of the dispensing mechanism 200 is activated, the first inlet opening 215 draws the first substance 310, such as anhydrous lyophilized probiotic bacteria matrix, through the first tube 210 toward the first outlet opening 217 to dispense the first substance 310 from the storage vessel 110. Additionally, as illustrated in FIGS. 3 and 4, the first tube 210 has a first protruding member 218 that is positioned toward the first tube first end 212 which extends away from the circumferential wall 216 of the first tube 210 and defines a first protruding member diameter 219 such that first protruding member diameter 219 is greater than the first tube diameter 213. Furthermore, the first protruding member 218 interfaces with a third inlet opening 268 of the dispensing head 250 such that once the dispensing head 250 is activated by a user, the first protruding member 218 allows the first tube 210 and the dispensing head 250 to draw the first substance 310 from the first inlet opening, toward the first outlet opening 217, to the third inlet opening 268, and out a third outlet opening 272.

Similar to the first tube 210, the second tube 220 has a second tube first end 222, a second tube second end (not illustrated), a length that is measured from the second tube first end 222 to the second tube second end, a second tube first diameter 223, and a circumferential wall 226 that extends between the second tube first and second ends 222, 224. The second tube 220 also defines a second inlet opening (not illustrated) and a second outlet opening 227 in which the second inlet opening is disposed at the second tube second end, and the second outlet opening 227 is disposed at the second tube first end 222. As described in more detail below, the second inlet opening provides suction such that once the dispensing head 250 of the dispensing mechanism 200 is activated, the second inlet opening draws the second substance 350, such as a moisture-providing composition, through the second tube 220 toward the second outlet opening 227 to dispense the second substance 350 from the storage vessel 110. Additionally, as illustrated in FIGS. 3 and 4, the second tube 220 has a second protruding member 228 that is positioned toward the second tube first end 222 which extends away from the circumferential wall 226 of the second tube 220 and defines a second protruding member diameter 229 such that second protruding member diameter 229 is greater than the second tube diameter 223. Furthermore, the second protruding member 228 interfaces with the fourth inlet opening 272 of the dispensing head 250 such that once the dispensing head 250 is activated by a user, the second protruding member 228 allows the second tube 220 and the dispensing head 250 to draw the second substance 350 from the second inlet opening, toward the second outlet opening 227, to the fourth inlet opening 270, and out a fourth outlet opening 274.

The dispensing mechanism 200 also includes first and second collars 230A, 230B. As illustrated in FIG. 3, the first and second collars 230A, 230B are attached to each of the first and second openings 146A, 146B of the first stopper 130 such that the first collar 230A is attached to the first openings 146A, and the second collar 230B is attached to the second openings 146B. Furthermore, each of the first and second collars 230A, 240B defines a threading (not illustrated) that is complementary to each threading of the first and second openings 146A, 146B such that each of the first and second collars 230A, 230B are attached and secured to each of the first and second openings 146A, 146B of the first stopper 130, which is described above. Thus, each attachment between the first and second collars 230A, 230B and the first and second openings 146A, 146B of the first stopper 130 is considered advantageous at least because each attachment prevents movement between each of the first and second chambers 170, 180 and between each of the first and second tubes 210, 220 of the dispensing mechanism 200.

Additionally, each of the first and second springs 240A, 240B of the dispensing mechanism 200 is positioned over a portion of the first and second tubes 210, 220. As illustrated in FIG. 4, the first spring 240A has a first spring first end 242A and a first spring second end (not illustrated), and the first spring 240A is positioned along a portion of the circumferential wall 216 of the first tube 210 toward the first tube second end 214. The first spring first end 242A interfaces with the first protruding member 218, and the first spring second end cooperatively interfaces with the first collar 230A, the first opening 146A of the first stopper 130, and the first chamber 170. During use, the first spring 240A of the dispensing mechanism 200 compresses from a first position to a second position once a user exerts force on the dispensing head 250 such that the dispensing head 250 transition from the storage vessel first end 112 toward the storage vessel second end 114. Here, the first spring 240A allows the dispensing head 250 to transition from a first position to a second position when compressed. Moreover, once the user removes the force from the dispensing head 250, the first spring 240A decompresses and exerts a force that expands the first spring 240A from the second position to the first position and allows the dispensing head 250 to transition to its first position such that the dispensing head 250 transitions toward the storage vessel first end 212.

Similarly, as illustrated in FIG. 4, the second spring 240B has a second spring first end 242B and a second spring second end (not illustrated), and the second spring 240B is positioned along a portion of the circumferential wall 226 of the second tube 220 toward the second tube second end 224. The second spring first end 242B interfaces with the second protruding member 228, and the second spring second end cooperatively interfaces with the first collar 230B, the second opening 146B of the first stopper 130, and the second chamber 180. During use, the second spring 240B of the dispensing mechanism 200 compresses from a first position to a second position once a user exerts force on the dispensing head 250 such that the dispensing head 250 transition from the storage vessel first end 112 toward the storage vessel second end 114. Here, the second spring 240B allows the dispensing head 250 to transition from a first position to a second position when compressed. Moreover, once the user removes the force from the dispensing head 250, the second spring 240B decompresses and exerts a force that expands the second spring 240B from a second position to a first position and allows the dispensing head 250 to transition to its first position such that the dispensing head 250 transitions toward the storage vessel first end 212.

As illustrated in FIGS. 2, 5, and 6, the dispensing head 250 has a dispensing head first end 252, a dispensing head second end 254, a dispensing head third end 256, a dispensing fourth end 258, a dispensing head inner surface 251, a dispensing head outer surface 253, a dispensing head main opening 260, a first passageway 262, a second passageway 264, a third passageway 266, and a second set of attachment members 276. In the illustrated embodiment, the dispensing head first and second ends 252, 254 directly oppose each other, and the dispensing head third and fourth ends 256, 258 directly oppose each other.

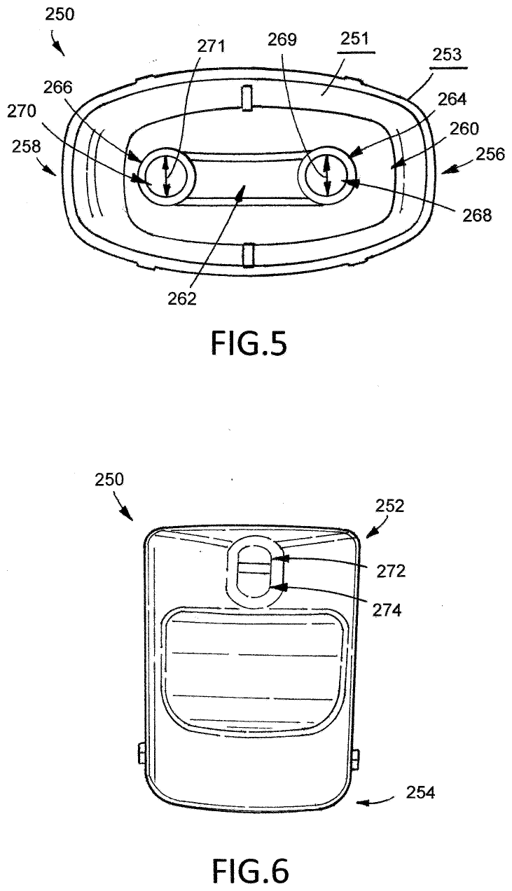

As illustrated in FIG. 5, the dispensing head main opening 260 extends from the dispensing head second end 254 toward the dispensing head first end 252 and is disposed between the dispensing head third and fourth ends 256, 258. The dispensing head 250 also defines a first passageway 262, a second passageway 264, and a third passageway 266. In the illustrated embodiment, each of the first, second and third passageways 262, 264, 266 is disposed inside of the dispensing head main opening 260. The first passageway 262 extends from the dispensing head fourth end 258 toward the dispensing head third end 256 and is positioned on the dispensing head inner surface 251 on the dispensing head first end 252. The second passageway 264 is connected to the first passageway 262 such that the second passageway 264 is continuous with the first passageway 262, and the second passageway 264 is positioned toward the dispensing head third end 256 and extends from the first passageway 264 toward the dispensing head second end 254. Similarly, the third passageway 266 is connected to the first passageway 262 such that the third passageway 266 is continuous with the first passageway 262, and the third passageway 266 is positioned toward the dispensing head fourth end 258 and extends from the first passageway 264 toward the dispensing head second end 254. Moreover, as illustrated in FIGS. 5 and 6, each of the second and third passageways 264, 266 defines each of the third and fourth inlet openings 268, 270 at the dispensing head second end 254 such that the second passageway 264 defines the third inlet opening 268 and the third passageway 266 defines the fourth inlet opening 270. Furthermore, the first passageway 262 defines each of the third and fourth outlet openings 272, 274 at the dispensing head fourth end 258.

In the illustrated embodiment, the third and fourth inlet openings 268, 270 are sized and configured to cooperatively interface with the first and second outlets 217, 227 and the first and second protruding members 218, 228 of the first and second tubes 210, 220. As such, each of the third and fourth inlet openings 268, 270 defines a diameter such that the third inlet 268 defines a third inlet diameter 269 and the fourth inlet 270 defines a fourth inlet diameter 271. The third and fourth inlet diameters 269, 271 are also continuous along each of the second and third passageways. Additionally, each of the third and fourth inlet diameters 269, 271 is greater than the first and second tube diameters 213, 223. This structural configuration is considered advantageous at least because a portion of each of the first and second tubes 210, 220 may be inserted into a portion of each of the first and second passageways 264, 266 to adequately dispense the first and second substances. However, each of the third and fourth inlet diameters 269, 271 is less than the first and second protruding members diameters 219, 229. This structural configuration is considered advantageous at least because the first and second protruding members 218, 228 allows the dispensing head 250 to adequately engage each of the first and second tubes 210, 220 and to adequately dispense each of the first and second substances 310, 350 at a predetermined amount.

While the second and third passageways 264, 266 are connected and continuous with the first passageway 262 to dispense the first and second substances 310, 350, the first passageway 262 may be omitted from the dispensing head 250 such that the second and third passageways 264, 266 are independent of each other and each individually dispenses either the first or second substances 310, 350. In this alternative embodiment, the second passageway 264 would extend between the third inlet 268 to the third outlet 272, and the third passageway 266 would extend between the fourth inlet 270 to the fourth outlet 274. Thus, the first and second substances 310, 350 would be dispensed separately such that the first and second substances 310, 350 would be mixed together by the user, not by the dispensing mechanism 200. The omission of the first passageway 262 and the separation of the second and third passageways 264, 266 are considered advantageous at least because the structural configuration between each of the second and third passageways 264, 266 prevents the mixture of the first and second substances 310, 350 prior to the user applying the mixture to his or her skin.

The dispensing mechanism 200, as illustrated and described herein, is considered advantageous at least because the dispensing mechanism allows both first and second substances 310, 350 to be adequately mixed together. Once a user engages the dispensing head 250 and each substance is drawn through each of the first and second tubes 210, 220, the first and second substances 310, 350 are mixed inside of the first passageway 262 once each of the first and second substances 310, 350 exits each of the second and third passageways 264, 266. However, if the second and third passageways 264, 266 are independent of each other, the first and second substances 310, 350 will not be mixed together during dispensing but rather mixed by the user.

In addition, the packaged probiotic composition 100 also includes a cap 190. The cap 190 defines a cap first end 192, a cap second end 194, and a cap inner surface 191. The cap inner surface 191 defines a fourth set of attachment members 194 located at the cap second end 194. The fourth set of attachment members 194 are sized and configured to interface and attach to the third set of attachment member 137 of the first stopper 130 in order for the cap 190 to remain fix to the storage vessel 110, which is described above.

In this embodiment, the first substance 310 packaged in the first chamber 170 of the packaged probiotic composition 100 is a lyophilized probiotic bacteria matrix composition. The lyophilized probiotic bacteria matrix composition provides production of compounds in any one of accelerating the healing of the skin, increasing collagen and elastin production, reducing and/or preventing skin inflammation, and producing antibacterial agents to reduce skin irregularities, and inhibiting skin-altering compounds and mechanisms against pathogenic bacterium such as Propionibacterium acnes (P. acnes), Staphylococcus aureus (Staph), and other similar pathogenic bacterium. The lyophilized probiotic bacteria matrix composition also includes an anhydrous mixture that has a probiotic bacterium for topical administration to the user's skin with the second substance 350, as described below. In addition, the bacteria of the lyophilized probiotic bacteria matrix composition comprises at least Lactobacillus rhamnosus. Moreover, the strain of bacteria of the Lactobacillus rhamnosus includes at least a TP40 strain.

Furthermore, in this embodiment, the second substance 350 packaged in the second chamber 180 of the packaged probiotic composition 100 is a moisture-providing composition. The moisture-providing composition provides any one of hydrating to the skin, reducing and/or preventing fine lines around the eyes and lips, reducing and/or preventing wrinkles to the skin, treating acne and rosacea, increasing skin thickness, and smoothing rough skin. In this embodiment, the moisture-providing composition includes a moisturizer that comprises antioxidants. In this embodiment, the moisture-providing composition includes at least Vitamin A, E, C and K, Hyaluronic acid, Biotin, Niacinamide, Resveratrol, dimethylaminoethanol (DMAE), PCA, Palmitoyl Tripeptide-1, Palmitoyl Tetrapeptide-7, Ceramides, Alpha-Lipoic Acid, Malic Acid, and Coenzyme Q10 (CoQ10) which can be topically administered to the user's skin in combination with the first substance 310, as described above.

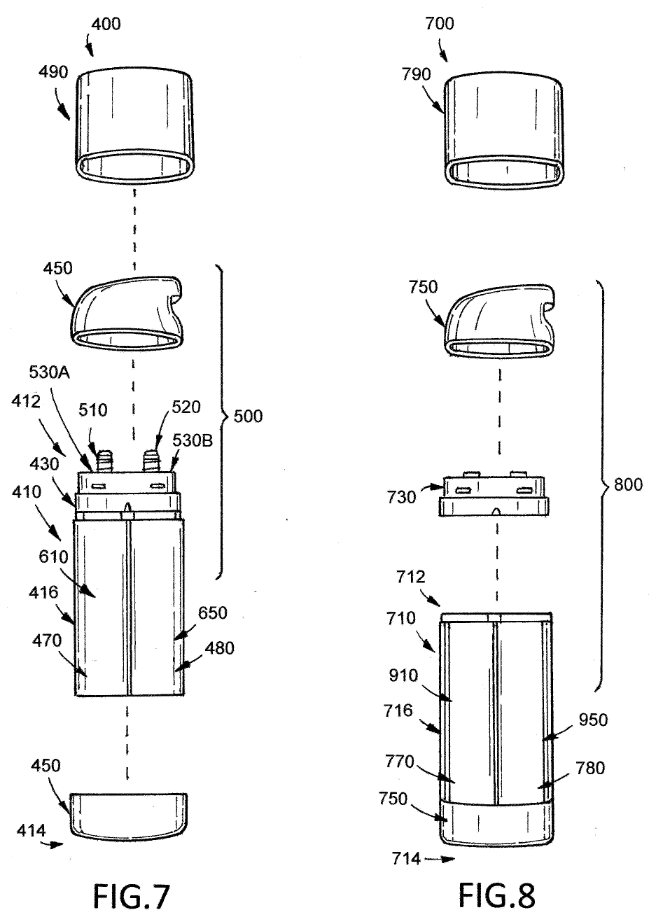

FIG. 7 illustrates another example packaged probiotic composition 400 that includes a storage vessel 410, a cap 490, and a dispensing mechanism 500. The packaged probiotic composition 400 is similar to the packaged probiotic composition 100 described above, except as detailed below.

As illustrated in FIG. 7, the storage vessel 410 has a storage vessel first end 412, a storage vessel second end 414, a main body 416, a first stopper 430, a second stopper 450, a first chamber 470, and a second chamber 480. Additionally, the dispensing mechanism 500 includes a first tube 510, a second tube 520, first and second collars 530A, 530B, and a dispensing head 550. In this illustrated embodiment, however, each of the first and/or second chamber 470, 480 is removable such that either the first chamber 470, the second chamber 480, or both first and second chambers 470, 480 may be removed from the storage vessel 410 and the dispensing mechanism 500. A first substance, such as an anhydrous lyophilized probiotic bacteria matrix, as described above, is disposed in the first chamber 470. Similarly, a second substance, such as a moisture-providing composition, as described above, is disposed in the second chamber 480.

In the illustrated embodiment, each of the first and second chambers 470, 480 are removable from the storage vessel 410. In one example, the second stopper 450 of the storage vessel 410 is removable from the main body 416 such that either the first and/or second chambers 470, 480 may be readily removable by a user. The attachment between the second stopper 450 and the main body 416 is considered advantageous at least because a user may easily remove each of the first and/or second chambers 470, 480 if one of the first or second chambers 470, 480 does not contain either first or second substances 610, 650. Thus, the user may easily exchange a depleted first and/or second chamber 470, 480 for a first or second chamber that contains first or second substances 610, 650. A skilled artisan will be able to select an appropriate structural attachment between the second stopper 450 and the main body 416 according to an embodiment based on various considerations, including the size of each chamber and the number of chambers that may be removed from the storage vessel. Examples of suitable structural attachments between a second stopper and a main body include a second stopper releasably attached to a main body, a second stopper integrally attached to a main body, a second stopper hingedly attached to a main body, a second stopper slideably attached to a main body, and other suitable structural attachments between a second stopper and a main body.

If the packaged probiotic composition 400 includes a removable second stopper 450 in order to remove the first and/or second chambers 470, 480, the user may first remove the second stopper 450 from the main body 416. Once removed, the user may remove the first and/or second collars 530A, 530B from the first and/or second openings (not illustrated) from the first stopper 430 depending whether the first and/or second chambers 470, 480 will be removed. The first or second collars 530A, 530B are removed from the first stopper 430 and moved toward the storage vessel first end 412 and away from the main body 416 to completely disconnect the first and/or second collars 530A, 530B from the storage vessel 410 and the dispensing mechanism 500. The first or second collars 530A, 530B are also removed from the first or second tubes 510, 520 due to the first and second tubes 510, 520 remaining with the first and/or second chambers 470, 480. Once the first and/or second collars 530A, 530B are removed, the user may remove the first and/or second chambers 470, 480 from the storage vessel 410. The user may remove each of the first and/or second chambers 470, 480 away from the storage vessel 410 by exerting a pulling force toward the storage vessel second end 414 to completely disconnect each of the first and/or second chambers 470, 480. Once the first and/or second chambers 470, 480 are removed, the user may insert new, first and/or second chambers 470, 480 into the storage vessel 410 by performing the previous steps in the reverse order.

FIG. 8 illustrates another example packaged probiotic composition 700 that includes a storage vessel 710, a cap 790, and a dispensing mechanism 800. The packaged probiotic composition 700 is similar to the packaged probiotic composition 100 described above, except as detailed below.

As illustrated in FIG. 8, the storage vessel 710 has a storage vessel first end 712, a storage vessel second end 714, a main body 716, a first stopper 730, a second stopper 750, a first chamber 770, and a second chamber 780. Additionally, the dispensing mechanism 700 includes a first tube 710, a second tube 720, first and second collars 730A, 730B, and a dispensing head 850. In this illustrated embodiment, however, each of the first and/or second chamber 770, 780 is removable such that either the first chamber 770, the second chamber 780, or both first and second chambers 770, 780 may be removed from the storage vessel 710 and the dispensing mechanism 800. A first substance, such as an anhydrous lyophilized probiotic bacteria matrix, as described above, is disposed in the first chamber 770. Similarly, a second substance, such as a moisture-providing composition, as described above, is disposed in the second chamber 780.

The first stopper 730 of the storage vessel 710 may be removable from the main body 716 such that either the first and/or second chambers 770, 780 may be readily removable by a user. The attachment between the first stopper 730 and the main body 716 is considered advantageous at least because a user may easily remove each of the first and/or second chambers 770, 780 if one of the first or second chambers 770, 780 does not contain either first or second substances 910, 950. Thus, the user may easily exchange a depleted first and/or second chamber 770, 780 for a first or second chamber 770, 780 that contains first or second substances 910, 950. A skilled artisan will be able to select an appropriate structural attachment between the first stopper 730 and the main body 716 according to an embodiment based on various considerations, including the size of each chamber and the number of chambers that may be removed from the storage vessel. Examples of structural attachment between a first stopper and a main body include a first stopper releasably attached to a main body, a first stopper integrally attached to a main body, a first stopper hingedly attached to a main body, a first stopper slideably attached to a main body, and other suitable structural attachments between a first stopper and a main body.

If the packaged probiotic composition 700 includes a first stopper 730 that is removable, the user may remove the first and/or second collars (not illustrated) from the first and/or second openings (not illustrated) from the first stopper 730 depending whether the first and/or second chambers 770, 780 will be removed. The first or second collars are removed from the first stopper 730 and moved toward the storage vessel first end 712 and away from the main body 716 to completely disconnect the first and/or second collars from the storage vessel 710 and the dispensing mechanism 800. The first or second collars are also removed from the first or second tubes (not illustrated) due to the first and second tubes remaining with the first and/or second chambers 770, 780. Once the first and/or second collars are removed, the user may first remove the first stopper 730 from the main body 416. Once removed, the user may remove the first and/or second chambers 470, 480 from the storage vessel 710. The user may remove each of the first and/or second chambers 770, 780 away from the storage vessel 710 by exerting a pulling force toward the storage vessel first end 712 to completely disconnect each of the first and/or second chambers 770, 780. Once the first and/or second chambers 770, 780 are removed, the user may insert new first and/or second chambers 770, 780 into the storage vessel 710 by performing the previous steps in the reverse order.

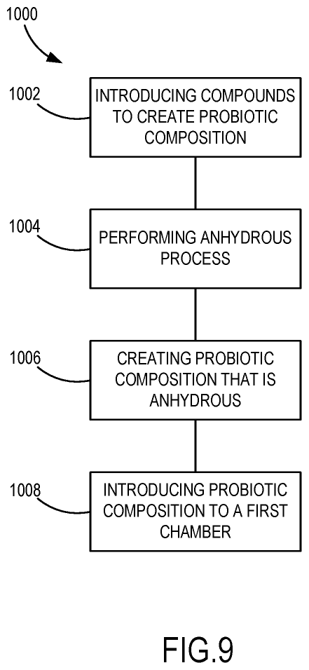

FIG. 9 illustrates a flowchart representation of a method 1000 of producing a packaged probiotic composition. Performance of the method 1000 produces a packaged probiotic composition according to an embodiment of the invention. The method is useful for producing one or several packaged probiotic compositions that may provide anti-aging and other beneficial properties, such as reduction of fine lines around the eyes and lips of a user, reduction and/or prevention of acne and/or rosacea, and increasing the production of the collagen and elastin in the skin of a user.

An initial step 1002 comprises producing the lyophilized probiotic bacteria matrix. The mixture to produce the lyophilized probiotic bacteria matrix composition comprises dimethicone, polysilicone-11, and butyrosperum parkii butter. The lyophilized probiotic bacteria matrix also comprises the bacteria Lactobacillus rhamnosus with a strain of TP40.

Another step 1004 comprises treating the lyophilized probiotic bacteria matrix composition such that the lyophilized probiotic bacteria matrix composition is anhydrous. This step can be accomplished by using any suitable technique or process for creating a compound that is anhydrous, such as using suitable drying process in conjunction with drying agents in dry conditions.

Another step 1006 comprises producing the lyophilized probiotic bacteria matrix composition that is anhydrous. This step can be accomplished once the lyophilized probiotic bacteria matrix composition is substantially free of water or crystalized water molecules.

Another step 1008 comprises placing and packaging a lyophilized probiotic bacteria matrix composition into a first chamber that is substantially free of air, as described above. This step can be accomplished by using any suitable technique in which the lyophilized probiotic bacteria matrix composition is packaged in an airless container to preserve the probiotic bacteria present in the composition. In addition, the lyophilized probiotic bacteria matrix composition is separate from a moisture-providing composition that is placed and packaged in a second chamber that is substantially free of air, as described above.

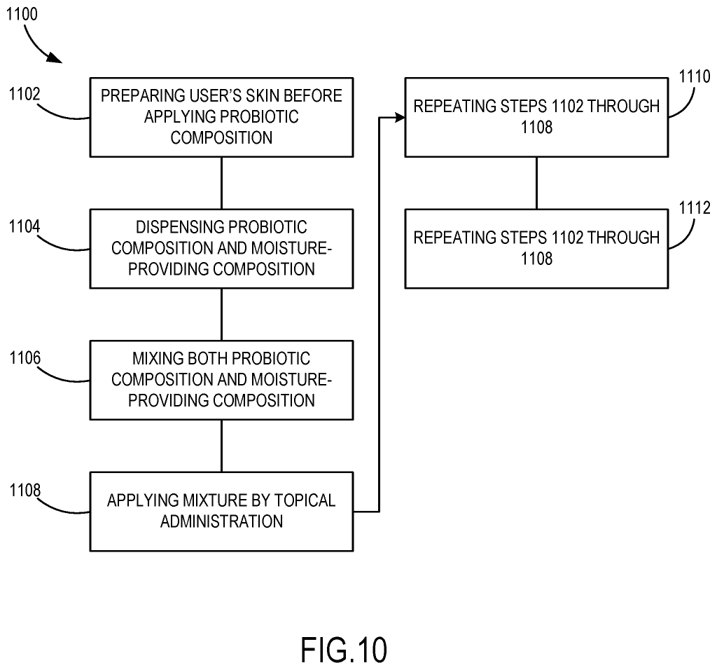

FIG. 10 illustrates a flowchart representation of a cosmetic method 1100 of using a packaged probiotic composition, such as packaged probiotic composition 100, 400, 700 that comprises both a lyophilized probiotic bacteria matrix that is anhydrous and a moisture-providing composition. Performance of the method results in administering a mixture of a lyophilized probiotic bacteria matrix that is anhydrous and a moisture-providing composition together. With this method, it is expected that the mixture of the lyophilized probiotic bacteria matrix that is anhydrous and the moisture-providing composition together provides anti-aging components to a user's skin.

The cosmetic method of applying the anhydrous lyophilized probiotic bacteria matrix composition and the moisture-providing composition to skin is considered advantageous at least because the mixture of the two composition provides an active probiotic culture with active ingredients to preserve and heal a user's skin microbiome. The mixture of the anhydrous lyophilized probiotic bacteria matrix composition and the moisture-providing composition provides antioxidants to help protect a user's skin against every day pollutants and moisturizes to help reduce and/or prevent fine line around a user's eyes and lips. The mixture of the anhydrous lyophilized probiotic bacteria matrix composition and the moisture-providing composition also provides support to underlying connective tissues and immune functions of a user such that mixture of the anhydrous lyophilized probiotic bacteria matrix composition and the moisture-providing composition increases in production of collagen and elastin. Moreover, mixture of the anhydrous lyophilized probiotic bacteria matrix composition and the moisture-providing composition reduces inflammation of the user's skin and accelerates the healing of the user's skin once administered.

Initial step 1102 comprises a user to prepare user's skin by adequately washing, scrubbing, or applying other medication, such as acne medication or other topical medication, before applying the mixture of the anhydrous lyophilized probiotic bacteria matrix composition and the moisture-providing composition to a localized area.

Step 1104 comprises using a packaged probiotic composition, such as packaged probiotic composition 100, 400, and 700. This step is accomplished by activating a dispensing mechanism of the probiotic composition dispenser, such as dispensing mechanism 200, 500, 800. To activate the dispensing mechanism, a user exerts force onto a dispensing head such that the user exerts a pushing force that moves the dispensing head from a first position to a second position. Once the dispensing head reaches its second positions, each of the anhydrous lyophilized probiotic bacteria matrix composition and moisture-providing composition packaged in first and second chambers are dispensed in a predetermined amount.

Another step 1106 comprises is a user to manually mix the predetermined amounts of the anhydrous lyophilized probiotic bacteria matrix composition and moisture-providing composition together. The mixing of the anhydrous lyophilized probiotic bacteria matrix composition and moisture-providing composition is accomplished by the user simply mixing both composition in the user's hands. An optional step for mixing the anhydrous lyophilized probiotic bacteria matrix composition and moisture-providing composition together is by the user mixing and rubbing both compositions into the skin at a desired location. This step may be omitted if the probiotic composition dispenser comprises a structural configuration that mixes the predetermined amounts of anhydrous lyophilized probiotic bacteria matrix composition and moisture-providing composition together before dispensing to a user. The mixture would occur within the dispensing mechanism, specifically within the dispensing head.

Another step 1108 comprises a user topically applying and/or administering the mixture of the anhydrous lyophilized probiotic bacteria matrix composition and moisture-providing composition to a localized area. This step is accomplished by the user simply applying the mixture of the compositions to an area of skin, such as a user's face, arm, hands, and other suitable areas that would benefit from the mixture of the anhydrous lyophilized probiotic bacteria matrix composition and the moisture-providing composition.

Another step 1110 comprises a user to repeat steps 1102 through 1108. Step 1110 is accomplished by repeating steps 1102 through 1108 at a second time on the same day after step 1108 is completed.

Another step 1112 comprises a user to repeat steps 1102 through 1108 after step 1110. Step 1112 is accomplished by repeating steps 1102 through 1108 at a third time on the same day after step 1110 is completed.

An optional step for the cosmetic method 1100 comprises treating and/or preventing acne or rosacea by topically administering the mixture of the anhydrous lyophilized probiotic bacteria matrix composition and the moisture-providing composition to a localized area. Another optional step for the cosmetic method 1100 comprises decreasing and/or preventing fine lines and wrinkles by topically administering the mixture of the anhydrous lyophilized probiotic bacteria matrix composition and the moisture-providing composition to a localized area, specifically to the face of a user. Another optional step for the cosmetic method 1100 comprises the maintaining of hydration of the skin by topically administering the mixture of the anhydrous lyophilized probiotic bacteria matrix composition and the moisture-providing composition to a localized area. Anther optional step for the cosmetic method 1100 comprises reducing and/or preventing skin inflammation by topically administering the mixture of the anhydrous lyophilized probiotic bacteria matrix composition and the moisture-providing composition to a localized area. Another optional step for cosmetic method 1100 comprises increasing production of collagen and elastin by topically administering the mixture of the anhydrous lyophilized probiotic bacteria matrix composition and the moisture-providing composition to a localized area.

The foregoing detailed description refers to various example probiotic composition dispensers and methods of producing probiotic composition and cosmetic uses of the probiotic composition. The description and appended drawings illustrating the probiotic composition dispensers and methods are intended to only provide examples and not to limit the scope of the claims in any manner.

We claim:

1. A packaged probiotic composition, comprising:
a storage vessel defining a first chamber, a second chamber, and a wall separating the first and second chambers;
a dispensing mechanism individually coupled to the first and second chambers;
a lyophilized probiotic bacteria matrix disposed in the first chamber;
a moisture-providing composition disposed in the second chamber; and
wherein activation of the dispensing mechanism draws pre-measured portions of the lyophilized probiotic bacteria matrix and the moisture-providing composition from the respective first and second chambers, mixes the pre-measured portions, and dispenses the resulting mixed composition.

2. The packaged probiotic composition of claim 1, wherein the first chamber and second chambers are substantially free of air.

3. The packaged probiotic composition of claim 1, wherein the lyophilized probiotic bacteria matrix comprises a bacteria of Lactobacillus rhamnosus of the strain TP40.

4. The packaged probiotic composition of claim 1, wherein the lyophilized probiotic bacteria matrix is anhydrous.

5. The packaged probiotic composition of claim 1, wherein the storage vessel comprises a first stopper and a second stopper; and
wherein the first and second stopper remain fixed to the storage vessel.

6. The packaged probiotic composition of claim 6, wherein the first and second chambers remain fixed inside of the storage vessel.

7. The packaged probiotic composition of claim 1, wherein the first and second chambers are movable such that each of the first and second chambers can be separably removed from the storage vessel.

8. The packaged probiotic composition of claim 8, wherein the storage vessel comprises a first stopper and a second stopper; and
wherein the first or second stopper is releasably attached to the storage vessel.

9. The packaged probiotic composition of claim 8, wherein the dispensing mechanism comprises a dispensing head including a first passageway, a second passageway, and a third passageway; and
wherein the first passageway is in fluid communication with the first chamber, the second passageway is in fluid communication with the second chamber, and third passageway is in fluid communication with the first and second passageway to substantially mix the lyophilized probiotic bacteria matrix and the moisture-providing composition together when dispensed.

10. The packaged probiotic composition of claim 1, wherein the dispensing mechanism comprises a dispensing head including a first passageway and a second passageway; and
wherein the first passageway is in fluid communication with the first chamber, the second passageway is in fluid communication with the second chamber, and first and second passageways independently dispense the lyophilized probiotic bacteria matrix and the moisture-providing composition without mixing.

11. A packaged probiotic composition, comprising:
a storage vessel defining a first chamber, a second chamber, and a wall separating the first and second chambers, each of the first and second chambers are substantially free of air;
a dispensing mechanism individually coupled to the first and second chambers;
a lyophilized probiotic bacteria matrix comprising Lactobacillus rhamnosus disposed in the first chamber;
a moisture-providing composition disposed in the second chamber; and
wherein activation of the dispensing mechanism draws pre-measured portions of the lyophilized probiotic bacteria matrix and the moisture-providing composition from the respective first and second chambers, mixes the pre-measured portions, and dispenses the resulting mixed composition.

12. The packaged probiotic composition of claim 11, wherein the lyophilized probiotic bacteria matrix is anhydrous.

13. The packaged probiotic composition of claim 11, wherein the storage vessel comprises a first stopper and a second stopper, and wherein the first and second stopper remain fixed to the storage vessel.

14. The packaged probiotic composition of claim 13, wherein the first and second chambers remain fixed inside of the storage vessel.

15. The packaged probiotic composition of claim 11, wherein the first and second chambers are movable such that each of the first and second chambers can be separable removed from the storage vessel.

16. The packaged probiotic composition of claim 15, wherein the storage vessel comprises a first stopper and a second stopper, and
wherein the first or second stopper is releasably attached to the storage vessel.

17. The packaged probiotic composition of claim 11, wherein the dispensing mechanism comprises a dispensing head including a first passageway, a second passageway, and a third passageway; and
wherein the first passageway is in fluid communication with the first chamber, the second passageway is in fluid communication with the second chamber, and third passageway is in fluid communication with the first and second passageway to substantially mix the lyophilized probiotic bacteria matrix and the moisture-providing composition together when dispensed.

18. The packaged probiotic composition of claim 11, wherein the dispensing mechanism comprises a dispensing head including a first passageway and a second passageway; and
wherein the first passageway is in fluid communication with the first chamber, the second passageway is in fluid communication with the second chamber, and first and second passageways independently dispense the lyophilized probiotic bacteria matrix and the moisture-providing composition without mixing.

19. The packaged probiotic composition of claim 11, wherein the moisture-providing composition comprises at least at least Vitamin A, E, C and K, Hyaluronic acid, Biotin, Niacinamide, Resveratrol, dimethylaminoethanol, PCA, Palmitoyl Tripeptide-1, Palmitoyl Tetrapeptide-7, Ceramides, Alpha-Lipoic Acid, Malic Acid, and Coenzyme Q10.

20. A packaged probiotic composition, comprising:
a storage vessel defining a first chamber, a second chamber, and a wall separating the first and second chambers, each of the first and second chambers are substantially free of air;
a dispensing mechanism individually coupled to the first and second chambers;
a lyophilized probiotic bacteria disposed in the first chamber, the lyophilized probiotic bacteria matrix comprising Lactobacillus rhamnosus having the ingredients of dimethicone, polysilicone-11, and butyrospermum parkii butter;
a moisture-providing composition disposed in the second chamber, the moisture-providing composition at least the ingredients of Vitamin A, E, C and K, Hyaluronic acid, Biotin, Niacinamide, Resveratrol, dimethylaminoethanol, PCA, Palmitoyl Tripeptide-1, Palmitoyl Tetrapeptide-7, Ceramides, Alpha-Lipoic Acid, Malic Acid, and Coenzyme Q10; and
wherein activation of the dispensing mechanism draws pre-measured portions of the lyophilized probiotic bacteria matrix and the moisture-providing composition from the respective first and second chambers, mixes the pre-measured portions, and dispense the resulting mixed composition.
